(19) United States
(12) Patent Application Publication
(10) Pub. No.: US 2015/0080867 A1
(43) Pub. Date: Mar. 19, 2015

(54) Gastric Reflux Treatment with Lasers

(71) Applicants: Wolfgang Neuberger, Dubai (AE); Martin Sabado, Cambridge (GB)

(72) Inventors: Wolfgang Neuberger, Dubai (AE); Martin Sabado, Cambridge (GB)

(21) Appl. No.: 14/030,790

(22) Filed: Sep. 18, 2013

(51) Int. Cl. A61B 18/20

(52) U.S. Cl. CPC A61B 18/20
USPC 606/10

(57) ABSTRACT

Method/devices for endoscopic laser treatment of gastrointestinal disorders such as, gastro-esophageal reflux disorder (GERD) are disclosed. Treatment involves irradiating tissue of the digestive tract to strengthen muscle tone and shrink anatomical areas reducing occurrence of reflux of food/liquids from the stomach to the esophagus. In a preferred embodiment, equipment comprises a gastro-endoscope with a working channel for inserting an optical fiber. Laser device comprises a diode laser source emitting a suitable wavelength and wave pattern through optical fiber. Laser radiation is applied near the Z line at the esophago-gastric junction. Measures are taken to assure irradiation is very accurate. Injury to the mucosa and adjacent structures, e.g. nerves, are avoided by focusing at the muscularis layer while preventing thermal effects on mucosa. This is achieved using wavelengths of ca. 1470 nm or ca. 980 mm. Energy is emitted perpendicular to fiber axis using optical fibers with side fiber distal tips, radial fiber distal tips or twister fiber distal tips.

### CROSS REFERENCE TO PRIORITY APPLICATION

This patent application is a continuation and claims priority to, U.S. provisional patent application No. 61/702,890, filed Sep. 19, 2012, by Martin Sabado and Wolfgang Neuberger entitled “Gastric Reflux Treatment with Lasers”, which is hereby expressly incorporated by reference in its entirety as part of the present disclosure.

### BACKGROUND OF THE INVENTION

1. Field of the invention

The invention relates to the field of treatment of gastrointestinal disorders and in particular for endoscopic transoral treatment of gastro esophageal reflux disorder (GERD).

2. Information Disclosure Statement

Gastro esophageal reflux disorder (GERD) occurs when the lower esophageal sphincter (LES), which connects the esophagus to the stomach, is weak or relaxes, allowing stomach contents to reflux back into the esophagus. Patients with GERD often experience a sensation known as heartburn, described as a burning pain that spreads to the throat and neck. When suffering from GERD, this heartburn and/or acid regurgitation may occur more than twice a week as a result of delicate esophageal tissue coming into contact with harsh stomach acid. This condition may also present as chest pain, difficulty swallowing, hoarseness in the morning, tightness in the throat, or even dry cough.

Heartburn may be worsened by meals, lying down, bending over, or drinking acidic beverages. Long-term complications may also occur, which include bleeding and ulceration of the esophagus or esophageal stricture. Some patients with chronic GERD may develop Barrett's esophagus, a condition in which esophageal tissue cells become abnormally shaped and colored, and may ultimately lead to cancer. Additionally, untreated GERD may exacerbate chronic conditions such as asthma, pulmonary fibrosis, and chronic cough.

There are several theories regarding the etiology of GERD. Various foods associated with GERD include acidic fruits, chocolate, fried foods, caffeinated drinks, onions, mint flavorings, and tomato-based products. These so-called trigger foods can reduce the tone of the LES, thereby causing a backflow of stomach contents. Hiatal hernias, caused by a disorder in which the top of the stomach extends past the diaphragm, may also influence GERD. Normally, the diaphragm helps the LES keep stomach contents from entering the esophagus. When this partnership is compromised, reflux occurs. Additional factors that may predispose a person to developing GERD are pregnancy, obesity, smoking, and the use of alcohol. Many physicians agree that most cases of severe GERD are due to a compromised LES tone and hiatal hernias. Additionally, transient LES relaxations (TLESRs) may cause mild to moderate GERD. Normally after swallowing, a peristaltic wave propels food to the distal esophagus and LES relaxation occurs for several seconds. Finally, delayed gastric emptying may also contribute to GERD by stimulating TLESRs because of gastric distention.

Lifestyle changes focus on strengthening the LES and reducing or blocking the secretion of acid. Physician recommendations include elevating the head of the bed, sleeping on left side, eating small meals, avoiding high-fat meals, eating at least three hours before lying down, losing weight, wearing loose clothing, and avoiding certain foods, drinks and drugs that exacerbate symptoms.

If lifestyle modifications are insufficient, or if patients prefer a different approach, physicians may provide medication such as antacids, Histamine type 2 receptor antagonists (H2RAs), and proton pump inhibitors (PPI). However, these mentioned drugs commonly present side effects including dizziness, headache, diarrhea, nausea and vomiting. Furthermore, patients who are very overweight often do not respond well to medicine.

Thus, many cases require surgical intervention. Patients considered for surgery include those who experience failure of medical therapy or who have intolerance or noncompliance to PPI therapy. Surgery may also be used in young patients with moderate to severe GERD. There are two main procedures available for surgical treatment of GERD. They are the Nissen and Toupet fundoplications, aimed at wrapping the fundus of the stomach around the lower end of the esophagus and fastening it in place to make the lower esophageal sphincter less compliable increasing the LES pressure to keep food and acid from coming back up. This may be done as an open procedure or by laparoscopy. However, surgery may present complications after the first weeks of procedure such as esophageal hemorrhage, perforation and gastric volvulus. Additionally, laparoscopic fundoplication surgery can cause a patient to feel bloated and to have difficulty in swallowing (dysphagia). These side effects may last for 30 days or longer and may require a second surgery to fix. Furthermore, some patients may also need additional antireflux medication to achieve further symptom relief. Other complications include gas, diarrhea, and flatulence. Finally, serious side effects related to any surgery, such as infection and heart attack, may occur with open or laparoscopic fundoplication procedures.

Endoscopic techniques have been studied to avoid the drawbacks of invasive surgical procedures. Endoscopic suturing near the LES, creates a barrier to reflux. However, current suturing treatments can cause pain in the chest and/or abdomen as well as bleeding. They can also cause patient to feel bloated and have difficulty swallowing. These side effects can last for 30 days or longer. Furthermore, the procedure is contraindicated in obese patients and those who present large hiatal hernias, dysphagia, or esophagitis. Another inconvenience related to existing transoral approaches is that they require precise sewing techniques, due to the cross-sectional area of the equipment which make it hard to maneuver.

A less invasive approach is the Stretta procedure, which uses a balloon to deliver radiofrequency (RF) energy at the gastro esophageal junction. It is proposed that RF ablation decreases the number of transient lower esophageal sphincter relaxations. However, there are hazards involved with radiofrequency. For example, nerve tissue, can be damaged when working with this type of energy. Another drawback of RF methods is that they require contact be maintained between the RF electrode and the tissue wall thus energy is delivered only through such points of contact. Additionally, RF catheters and electrodes can be relatively complex and more expensive to manufacture than other treatment systems. Furthermore, patients wearing pacemakers or other electronic implantable devices are in potential danger due to risk of interference with such devices due to radiofrequency waves and therefore they are not viable candidates for use of this type of device. Finally, quite frequently, medication is still necessary even after the radiofrequency treatment.

As can be seen from procedures discussed in the prior art, a safe and effective method and device for treating gastric reflux remains elusive. There is thus a need for a safe and long-term effective option for treatment of GERD. The present invention addresses this need.

### OBJECTIVES AND BRIEF SUMMARY OF THE INVENTION

It is an object of the present invention to provide an improved method for treatment of gastrointestinal disorders.

It is another object of the present invention to provide a safe, non-invasive in-office treatment of gastro esophageal reflux disorder.

It is still another object of the present invention to use laser energy for treatment of gastro esophageal reflux disorder.

Briefly stated, a method and device for endoscopic laser treatment of gastrointestinal disorders such as gastro esophageal reflux disorder (GERD) is disclosed. Treatment involves irradiating tissue of the digestive tract to strengthen muscle tone and shrink anatomical areas that could influence the occurrence of reflux of food and liquids from the stomach back to the esophagus. In a preferred embodiment, equipment comprises a gastro endoscope with a working channel for inserting at least one optical fiber. Laser device preferably contains a diode laser source which emits at a suitable wavelength and wave pattern through an optical fiber. Laser radiation is applied near the Z line at the esophago-gastric junction. Measures are taken to assure irradiation is very accurate. Injury to the mucosa and adjacent structures such as nerves are avoided by focusing at the muscularis layer while preventing/minimizing the thermal effect on the mucosa. This is preferably achieved with a wavelength of about 1470 nm or about 980 mm. In preferred embodiments, energy is emitted perpendicular to the optical fiber axis by using side fibers, radial fibers or twister fibers.

The above, and other objects, features and advantages of the present invention will become apparent from the following description read in conjunction with the accompanying drawings.

### BRIEF DESCRIPTION OF FIGURES

FIG. 1—shows an anatomic sketch of area to be treated.

FIG. 2—depicts main components of a preferred embodiment of present invention.

FIG. 3—illustrates possible fiber tips that can be chosen for treatment

### DETAILED DESCRIPTION OF PREFERRED EMBODIMENTS

Present invention seeks to noninvasively treat patients with gastrointestinal disorders by applying laser energy under direct vision to strengthen esophagus sphincter's muscle tone and reduce its circumference and prevent stomach contents from frequently going back into the esophagus.

In particular, the method is effective for treating gastro esophageal reflux disorder (GERD) by applying laser energy at the gastro esophageal junction area causing shrinkage and tightening of muscle tissue, thus preventing reflux of stomach content to the esophagus.

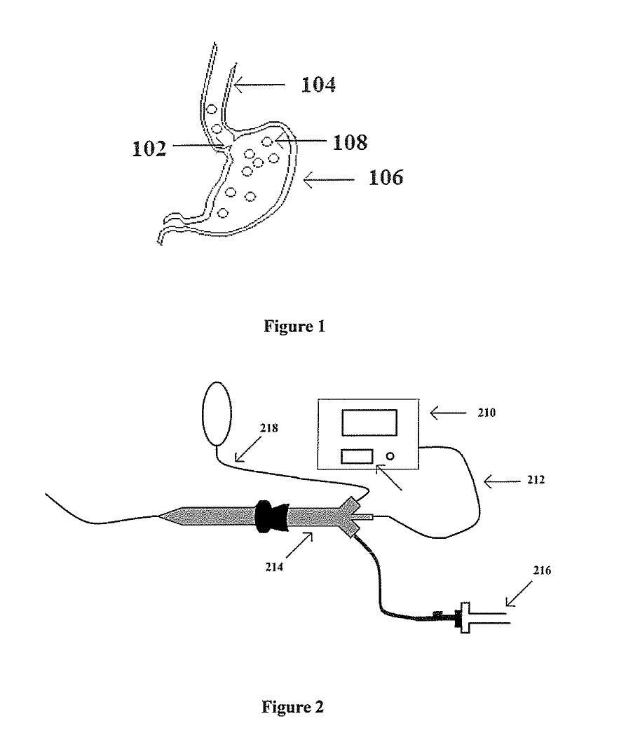

In FIG. 1, an anatomic sketch of the affected gastro esophageal area can be appreciated. Gastro esophageal reflux disorder (GERD) occurs when the lower esophageal sphincter (LES) 102, which connects the esophagus 104 to the stomach 106, is weak or relaxes, allowing stomach contents 108 to reflux into the esophagus 104. Patients with GERD often experience a sensation known as heartburn, described as a burning pain that spreads to the throat and neck. This heartburn and/or acid regurgitation occurs weekly as a result of delicate esophageal tissue coming into contact with harsh stomach acid. The present invention applies energy to the LES with the aim of inducing shrinkage of collagen tissue by causing thermal lesion, thus tightening LES muscle. After a healing period, LES muscle becomes tighter and stronger, thus reducing occurrence of gastric reflux.

Main components of a preferred embodiment of the present invention are illustrated in FIG. 2. Laser based system comprising diode laser source 210 emits at a suitable wavelength through fiber optics device 212 in a pattern according to chosen optical fiber distal tip. Possible fiber tips include but are not limited to radial, twister, side fiber, double ring radial. An endoscopic device 214 such as a gastroscope is used to assess correct placement of fiber optic. Optical fiber 212 is inserted through endoscopic device 214 to desired position. Imaging system. 216, an incorporated or a standalone imaging device, may be employed and may include but is not limited to ultrasound and camera vision. Additionally, viewing device allows for an on-line monitoring of the shrinking effect and for applying proper energy dosage into target tissue to prevent undesired consequences of under or overtreatment. For a clean, precise image, a flushing system 218 using ringer solution or medical grade CO2 is incorporated. Wavelength is preferably about 1470 nm.

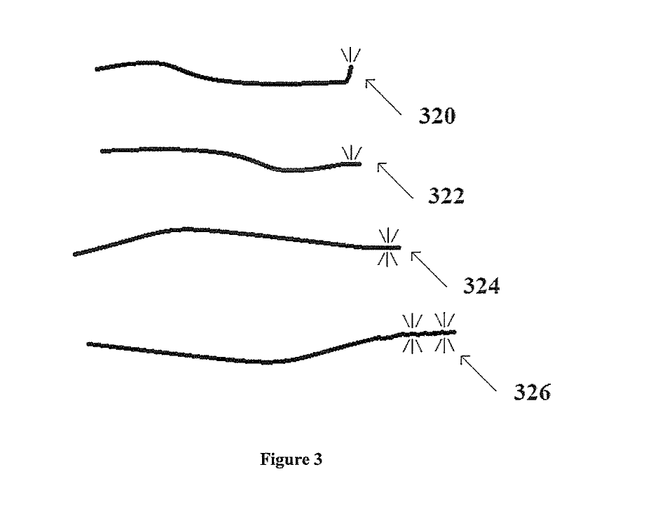

FIG. 3 depicts different optical fiber distal tips which a physician may choose from for various treatments. Possible fiber tips include twister fiber tips 320, side fiber tips 322, radial fiber tips 324 and double-ring radial fiber tips 326 according to the point or area that needs to be focused on in order to effectively achieve desired effect on muscle tissue, while avoiding injury by thermal effect of the mucosa and other structures such as nerves or other undesired effects. In a preferred embodiment, energy is applied only in certain points of the circumference of the esophageal lumen to avoid strictures or dysphagia.

In another preferred embodiment, a cylindrical guide piece is incorporated into system for centering optical fiber, thus allowing for more accurate radiation.

A preferred procedure for present invention comprises the following steps. Endoscopy is performed first to locate the gastro esophageal junction. Next, an optical fiber is introduced to just past the gastro esophageal junction or Z-line. Endoscope may be removed or left in place for online monitoring. Lasing parameters are set and laser energy then is delivered according to selected optic fiber tip. Finally, optic fiber is withdrawn and endoscopy is carried out once again to assess correct effect on the esophagus.

In a preferred embodiment, a side firing fiber or a twister fiber is selected. Energy is delivered longitudinally up to points separated at least 90 degrees from each other. In another preferred embodiment, a 360-degree radial emitting fiber is selected. Energy is delivered while moving fiber longitudinally. In another preferred embodiment, a double-ring radial fiber is selected. Lasing parameters are chosen and set such that energy delivered is absorbed by esophagus muscle layer and mucosa and penetration beyond this is minimized/absent.

In another embodiment, energy is delivered without moving the fiber during treatment. Thus, constriction in two locations at the esophago-gastric junction is achieved. Thermal energy induced by laser radiation causes collagen tissue to tighten and strengthen the sphincter. This strengthening of the LES helps prevent reflux of food content from the stomach back to the esophagus, thus eliminating GERD.

In another preferred embodiment, fiber is inserted inside an inflatable balloon filled with water or suitable cooling liquid to protect mucosa from overheating. A wavelength such as 980 nm or 810 nm is used.

The present invention is not merely limited to GERD. Other conditions related to managing muscle tone and/or size of the lower esophageal sphincter can be treated using the method of the present invention, such as morbid obesity.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A medical system for the treatment of gastric disorders comprising:
an endoscopic device;
at least one radiation source laser radiation device;
at least one fiber optic delivery means;
wherein said radiation source is capable of producing radiation at a preselected wavelength, a preselected wave pattern and a preselected power.

2. The medical system according to claim 1, wherein said preselected wavelength, said preselected wave pattern and said preselected power are chosen such that said radiation is absorbed by esophagus muscle tissue.

3. The medical system according to claim 1, wherein said preselected wavelength, said preselected wave pattern and said preselected power are chosen such that said radiation is absorbed by esophagus mucosa.

4. The medical system according to claim 1, further comprising a flushing system for better viewing.

5. The medical system according to claim 1, wherein said distal end of said fiber optic delivery means in chosen from the group consisting of twister fibers, side fibers, 360-degree radial fibers and double-ring radial fibers.

6. The medical system according to claim 1, further comprising a cylindrical piece for centering said optical delivery means.

7. A method for the treatment of gastric disorders comprising the steps of:
inserting an endoscopic device into the esophagus;
inserting a fiber optic delivery means into the esophagus to vicinity of target tissue;
setting radiation parameters;
applying radiation energy to target tissue;
removing fiber optic delivery means; and,
inserting endoscopic device to assess results achieved.

8. The method for treatment of gastric disorders according to claim 7, wherein fiber optic delivery means is chosen from the group of fibers having a distal tip of a side fiber tip, a twister fiber tip, a 360-degree radial fiber tip and a double-ring radial fiber tip.

9. The method for treatment of gastric disorders according to claim 7 wherein said fiber optic delivery means is inserted inside an inflatable balloon filled with cooling liquid.
